(19) United States
(12) Patent Application Publication
(10) Pub. No.: US 2006/0047121 A1
(43) Pub. Date: Mar. 2, 2006

(54) Novel process for preparation of clopidogrel bisulfate polymorph - Form I

(76) Inventors: Kamlesh Digambar Sawant, Mumbai (IN); Venkatasubramanian R. Tarur, Mumbai (IN); Dhananjay Govind Sathe, Thane (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 10/957,891

(22) Filed: Oct. 4, 2004

(30) Foreign Application Priority Data
Sep. 1, 2004 (IN) 945/MUM/2004

(51) Int. Cl. C07D 498/02

(52) U.S. Cl. 546/113

(57) ABSTRACT

A process for making Clopidogrel Bisulfate Form I which comprises dissolving Clopidogrel Bisulfate Form II in a solublizing solvent at room temperature to form a solution; adding an anti-solvent to the said solution till turbid; stirring the said turbid solution; collecting the precipitated solid and drying the final solid product, form I.

### RELATED APPLICATIONS

This application claims priority from India National patent application No. 945/MUM/2004, filed 1 Sep. 2004.

### GOVERNMENT INTEREST

None.

### TECHNICAL FIELD OF THE INVENTION

The present invention relates to a novel process for preparation of crystalline Form I of the antithrombotic agent Methyl (s)-(+)-α-(2-chlorophenyl)-4,5,6,7-tetrahydrothieno[2,3-c]pyridine-5-acetate hydrogen sulfate, commonly known as “clopidogrel bisulfate” (illustrated below as Formula I), from clopidogrel bisulfate Form II.

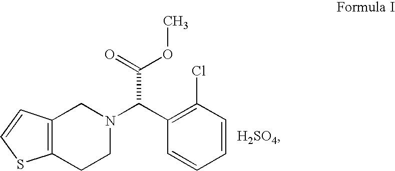

### BACKGROUND

Clopidogrel is administrated as its hydrogensulfate (syn. Bisulfate) salt. Its anti-platelet activity makes it an effective drug for reducing ischemic strokes, heart attacks and atherosclerosis, a vascular disease causing claudication. Atherosclerosis is a buildup of plaque in the walls of arteries, which leads to thickening of the arterial wall and reduction in the elasticity of the arteries. High Cholesterol, high blood pressure, smoking and infection also may cause injury to the inner walls of the arteries, which leads to the atherosclerosis. Plaque formation leads to blood clotting which is due to the platelet aggregation at the site of the injury. This clotting becomes an obstacle for the flow of the blood to the organs, causing heart attacks. Clopidogrel binds adenosine diphosphate to its receptor and thereby induces platelet reduction, which is desirable in fighting against the atherosclerosis.

The solid-state properties of the material (like flowability) affect the ease with which the material is handled during product formulation. Another important property is the rate of dissolution, which imposes a limit on the rate at which an orally administrated active ingredient can reach the blood stream. The solid state form of the compound can also affect its compaction behavior and its storage stability.

U.S. Pat. No. 4,847,265 describes the formation of the dextrorotatory isomer of clopidogrel by salt formation using a racemic compound mixture and an optically active acid (10-L-camphorsulfonic acid) in acetone, followed by successive recrystallisation until a product with constant rotatory power was obtained, followed by the release of the dextro rotatory isomer from its salt by a base. The hydrogen sulfate salt is then obtained by dissolution of the base in cold acetone, cooled in ice and addition of concentrated sulphuric acid to precipitate the bisulphate salt. The precipitate thus obtained is crystalline Form I.

U.S. Pat. No. 6,429,210 describes process for preparation of the dextrorotatory S enantiomer of Clopidogrel bisulfate in the crystalline Form, Form II. U.S. 2003114479 describes the novel crystalline forms, Form III, IV and V along with the process for preparation of Form I. In the '479 patent application, polymorphic Form I is prepared by suspending amorphous clopidogrel hydrogen sulphate in ether.

International patent application WO2004020443 describes process for preparation of Clopidogrel bisulfate Form I, which comprises separating out crystalline Form I from the solution of clopidogrel in the form of free base or salt in a solvent selected from the series of the primary, secondary or tertiary C1-C5 alcohols or their Esters with C1-C4 carboxylic acids or optionally of mixtures thereof.

International patent application WO 2004048385 describes a process for the preparation of crystalline Form I of S-Clopidogrel hydrogen sulphate by reacting the optically active base, (S)-(+) clopidogrel with concentrated sulfuric acid, wherein the salt formed by the said reaction in the reaction medium is precipitated with the precipitating solvent such as aliphatic or cyclic ethers and/or their mixture or isobutyl methyl ketone.

Form II of Clopidogrel Bisulfate is thermodynamically more stable. Hence, a small change in the reaction conditions during the preparation of Form I, can result in the formation of Form II instead.

The present invention relates to the process for preparation of the crystalline Form I of Clopidogrel bisulfate from Form II. The present invention also relates to a process for preparation of crystalline Form I of Clopidogrel Bisulfate, which is reproducible. The present invention also relates to a process for preparation of crystalline Form I of Clopidogrel Bisulfate, which is cost effective and economical. The present invention also relates to a process for preparation of crystalline Form I of Clopidogrel Bisulfate, which is commercially viable.

### SUMMARY OF THE INVENTION

This invention discloses a process for manufacturing crystalline Form I of Clopidogrel Bisulfate. Our process comprises crystallizing Form I from Form II by dissolving Form II in solvent (such as acetic acid) and then adding antisolvent (such as di-isopropyl ether).

### BRIEF DESCRIPTION OF THE FIGURES

FIG. 1 Shows the X-ray Diffraction Diagram of Clopidogrel Bisulfate Form I

FIG. 2 Shows the DSC Thermogram of Clopidogrel Bisulfate Form I

FIG. 3 Shows the FT-JR Spectrum of Clopidogrel Bisulfate Form I

### DETAILED DESCRIPTION

This invention discloses a process for manufacturing crystalline Form I of Clopidogrel Bisulfate, comprising crystallization or precipitation of Form I from Form II using solvent (such as a C₁ to C₄ carboxylic acid) and antisolvent (such as aliphatic ether). The present invention also relates to the solid-state form, Form I of Clopidogrel Bisulfate, made from this process.

The manufacturing process described in this invention involves crystallization of Form I from Form II in a reproducible way.

The said process comprises, for example,

Dissolving Clopidogrel Bisulfate Form II in glacial acetic acid at room temperature; Regenerating the clopidogrel bisulfate from the solution by adding an antisolvent to the solution at room temperature;

Stirring the reaction mixture for 24 hours at room temperature, filtering and drying the crystals to obtain Form I of Clopidogrel Bisulfate.

As used herein, a solvent is any liquid substance which has capacity to dissolve Clopidogrel Bisulfate Form II at room or higher temperature. “Antisolvent” is an organic solvent in which Clopidogrel Bisulfate has poor solubility.

As used herein, room temperature means a temperature from about 10° C. to about 45° C., preferably 25° C. to 30° C.

The Form II required for the preparation of Form I was prepared by the method described in U.S. Pat. No. 4,847,265.

The quality of clopidogrel bisulfate of Form I without detectable contamination by Form II, obtained in accordance with this invention, is documented by the following measurements:

X-ray powder diffraction pattern has been obtained on D 8-Advance, Bruker AXE, Germany, diffractometer equipped with Scintillation detector using Copper Kα (λ=1.5406 Å) radiation with scanning range between 2-50 θ at scanning speed of 2°/min.

Differential Scanning Calorimeter was performed on Mettler DSC 20 instrument. Samples of 2 mg to 3 mg weighed in aluminium crucibles with holes were scanned at a heating rate of I ° C. per minute under nitrogen atmosphere at rate of 35 ml/min.

The Fourier-transform infrared (FT-IR) spectrum of Form I was obtained on a FT-IR 8300, Shimadzu instrument, in the range of 400-4000 cm⁻¹ with a resolution of 4 m⁻¹.

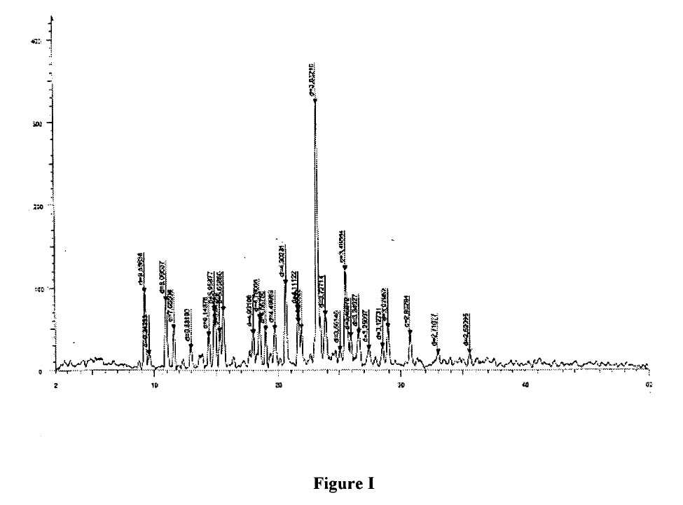

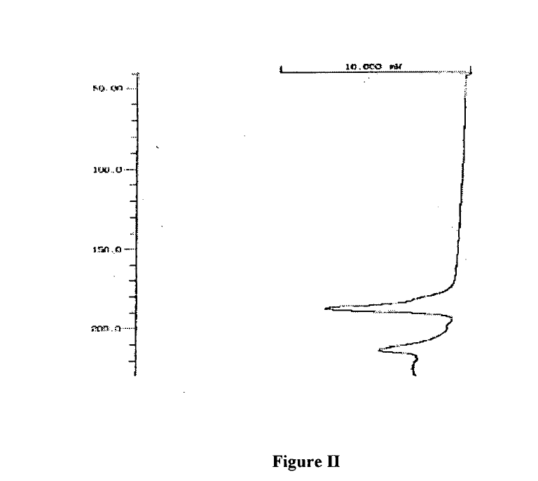

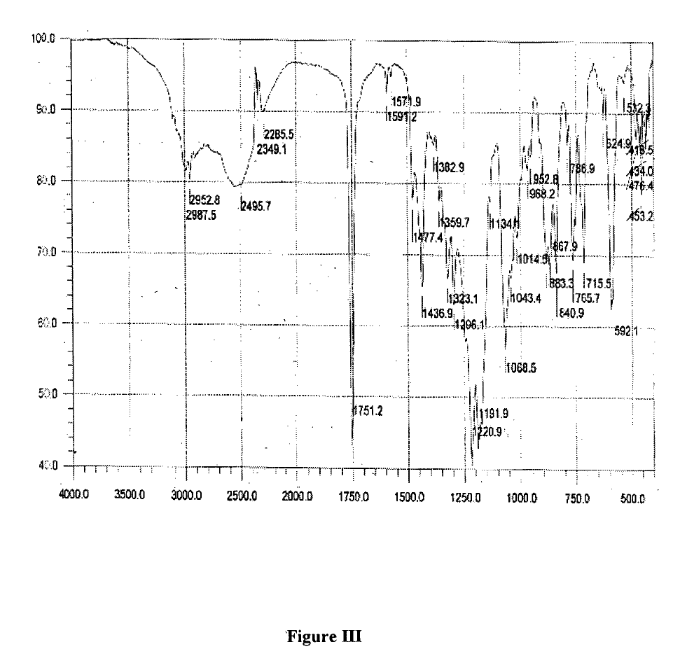

FIG. 1 shows an X-ray powder diffraction pattern of the polymorphic form (Form 1) obtained by this method. The X-ray powder diffraction pattern is characterized by having peaks at about 9.21, 9.56, 14.85, 15.53, 15.23, 20.62, 21.59, 23.19, 23.85, 25.52, ±0.2 degrees. FIG. 2 shows the DSC thermogram of the polymorphic form (Form I) obtained by this method. The DSC thermogram is characterized by having a sharp endotherm at about 187° C. and another sharp endotherm at about 212° C. FIG. 3 shows the FT-IR spectrum of the polymorphic form (Form I) obtained by this method. The FT-IR spectrum shows absorption at 2987, 2952, 1751, 1477, 1436, 1220, 1191, 867, 841, 766, 592 cm⁻¹.

The following examples are provided to illustrate the invention and are not intended to limit the scope of the appended claims.

### EXAMPLE 1

5 g. of Clopidogrel Bisulfate Form II was dissolved in 25 mL glacial acetic acid at room temperature. The solution was filtered to remove any suspended particles. To the resultant clear solution, 100 ml of diethyl ether was added, dropwise, at the same temperature. The solution was stirred for 24 hrs. at the same temperature. The solid was filtered, washed with diethyl ether and dried to get Form I.

### EXAMPLE 2

5 g. of crude Clopidogrel Bisulfate Form II was dissolved in 25 ml glacial acetic acid at room temperature. The solution was filtered to remove any suspended particles. To the resultant clear solution 100 ml of diisopropyl ether was added, dropwise, at the same temperature. The solution was stirred for 24 hrs. at the same temperature. The solid was filtered, washed with diisopropyl ether, and dried to get Form I.

Given our disclosure here, one of skill in the art may make variations and modifications. For example, while we use room temperature in our examples, we expect that one could run the same process at a higher temperature and obtain the same result. Thus, we intend the coverage of our patent to be defined by the claims alone, and not by the specific examples recited in the specification. For example, the Figures provide our actual laboratory data. They do not, however, limit the coverage of the appended claims. Thus, for example, our claims may cover product which does not have exactly the same X-ray diffraction pattern or DSC thermogram or FT-IR spectrum as is shown in the Figures.

We have defined certain claim terms in the Specification above. In the claims, we use the singular (e.g., “a” or “an” or “the”) to include the plural. Thus, for example, the claim phrase “comprising a solvent selected from the group consisting of A, B and C” literally encompasses (i) any one of the three enumerated solvents (alone or combined with a non-enumerated solvent), and (ii) two or more of the enumerated solvents (alone or together with a non-enumerated solvent).

1. A process comprising (a) dissolving Clopidogrel Bisulfate Form II in a solvent at room temperature to form a solution; (b) adding anti-solvent to said solution, said anti-solvent present in an amount sufficient to precipitate clopidrel bisulfate Form I from said solution; and (c) collecting precipitated clopidrel bisulfate Form I.

2. The process of claim 1 wherein said solvent comprises C₁-C₄ carboxylic acid.

3. The process of claim 1 wherein said room temperature is a temperature of from about 10° C. to about 45° C.

4. The process of claim 1 wherein said antisolvent comprises aliphatic ether.

5. The process of claim 1 wherein said antisolvent comprises aliphatic cyclic ether.

6. The process of claim 1, said collecting step comprising drying said precipitated clopidrel bisulfate Form I at a temperature of from about 50° C. to about 70° C.

7. The process of claim 3, wherein said room temperature is from about 25° C. to about 30° C.

8. The process of claim 2, wherein said C₁-C₄ carboxylic acid comprises acetic acid.

9. The process of claim 4, wherein said aliphatic ether is selected from the group consisting of: dimethyl ether; diethyl ether; and diisopropyl ether.

10. The process of claim 5, wherein said aliphatic cyclic ether is selected from the group consisting of: dioxane; and tetrahydrofuran.

11. A product comprising clopidrel bisulfate Form I made by the process of claim 1.

12. A product comprising clopidrel bisulfate Form I made by the process of claim 2.

13. A product comprising clopidrel bisulfate Form I made by the process of claim 3.

14. A product comprising clopidrel bisulfate Form I made by the process of claim 4.

15. A product comprising clopidrel bisulfate Form I made by the process of claim 5.

16. A product comprising clopidrel bisulfate Form I made by the process of claim 6.

17. A product comprising clopidrel bisulfate Form I made by the process of claim 1.

18. A product comprising clopidrel bisulfate Form I made by the process of claim 7.

19. A product comprising clopidrel bisulfate Form I made by the process of claim 8.

20. A product comprising clopidrel bisulfate Form I made by the process of claim 9.

21. A product comprising clopidrel bisulfate Form I made by the process of claim 10.

22. A method comprising administering to a human, a product comprising clopidrel bisulfate Form I made by the process of claim 1, in a cardiovascular therapeutic amount.
